(19) United States
(12) Patent Application Publication
(10) Pub. No.: US 2015/0258050 A1
(43) Pub. Date: Sep. 17, 2015

(54) METHOD FOR REDUCING TRIGLYCERIDES

(71) Applicant: DEAKIN UNIVERSITY, Waurn Ponds, Victoria (AU)

(72) Inventor: Andrew Sinclair, Point Lonsdale (AU)

(21) Appl. No.: 14/437,792

(22) PCT Filed: Oct. 23, 2013
(86) PCT No.: PCT/AU2013/001225

§ 371 Date: Apr. 22, 2015

Related U.S. Application Data
(60) Provisional application No. 61/717,157, filed on Oct. 23, 2012.

(51) Int. Cl. A61K 31/202; A23K 1/16; A23L 1/30

(52) U.S. Cl. CPC A61K 31/202; A23L 1/30; A23K 1/164; A23V 2002/00

(57) ABSTRACT

The present disclosure relates to docosapentaneoic acid (DPA) of the omega-3 type (DPA 22:5n-3) or derivative thereof and its use in reducing hypertriglyceridemia in a subject in need thereof. In particular, the disclosure relates to the ability of n-3 DPA to significantly decrease the incorporation of fatty acids in chylomicrons in the post-prandial setting.

### RELATED APPLICATIONS AND INCORPORATION BY REFERENCE

This application claims priority from U.S. 61/717,157 filed 23 Oct. 2012, the entire contents of which are herein incorporated by reference.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### FIELD OF THE INVENTION

The present disclosure relates to docosapentaneoic acid (DPA) of the omega-3 type (DPA 22:5n-3) or derivative thereof and its use in reducing hypertriglyceridemia in a subject in need thereof. In particular, the disclosure relates to the ability of n-3 DPA to significantly decrease the incorporation of fatty acids in chylomicrons in the post-prandial setting.

### BACKGROUND OF THE INVENTION

A vast amount of information exists in relation to the beneficial cardiovascular health actions of long-chain n-3 polyunsaturated fatty acids (n-3 PUFA), namely EPA and DHA. In contrast, little is known about the intermediary product docosapentaneoic acid (DPA 22:5 n-3), also known as 7, 10, 13, 16, 19 docosapentaenoic acid or clupanodonic acid.

The essential omega-3 type fatty acid alpha-linoleic acid (ALA, 18:3n-3) can be metabolized in vivo by elongation and desaturation enzymes to form a series of polyunsaturated fatty acids (PUFA) of the n-3 series. In addition to potentially being metabolized from ALA, eicosapentaenoic acid (EPA, 20:5n-3), docosahexaenoic acid (DHA, 22:6n-3) and docosapentaenoic acid (DPA, 22:5n-3) are provided from diet, mainly from fish and fish oil products, however the level of DPA is very low in fish oil.

Previous studies have demonstrated a significant elevation in the level of DPA in the circulating lipid fractions when human subjects have received seal oil (Conquer et al., (1999) Thromb Res 96,239-50, Meyer et al., (2009) Lipids 44, 827-35, Mann et al., (2010) Lipids 45, 669-681) as well as a significant rise in DPA concentrations in tissue lipids when animals have received seal oil (Murphy et al., (1999) Mol Cell Biochem 177, 257-69). However, such effects cannot be directly attributed to the consumption of DPA since it represents approximately 5% of the fatty acids in seal oil with a higher level of EPA which has a capacity to generate considerable amounts of DPA via chain elongation.

In rats, short term supplementation with pure DPA significantly increased the concentration of DHA in liver and the concentration of EPA in the liver, heart and skeletal muscle, presumably by the process of retroconversion (Kaur et al., (2010) Br J Nutr 130, 32-7). The apparent retroconversion from DPA to EPA especially in the kidney of the rats was also detected in recent studies by others.

In humans, knowledge regarding the post-prandial metabolism and biological effects of purified n-3 DPA is limited.

Given the recent global increase in the incidence of conditions associated with high levels of triglycerides, for example cardiovascular-related disease, obesity, and alcoholism, there is a need for improved treatments for these conditions.

### SUMMARY OF THE INVENTION

The present inventors sought to investigate and compare the postprandial metabolism of docosapentaenoic acid; DPA (22:5n-3) and eicosapentaenoic acid; EPA (20:5n-3) in human subjects following consumption of these fatty acids. Molecular level lipidomic analysis methods were used to investigate the structure and composition of the lipids in the human plasma with particular examination of metabolism of the n-3 polyunsaturated fatty acids (PUFA) in chylomicron triacylglycerols (TAG) and phospholipids.

The inventors surprisingly found that triglyceride levels in both the plasma and the chylomicron-rich fraction remained close to fasting levels after consumption of DPA alone and further, DPA did not raise the proportion of EPA in triglycerides. This finding strongly suggested that substantially purified DPA would be advantageous for lowering triglyceride levels in disorders associated with high plasma triglycerides, such as cardiovascular disorders, chylomicronemia syndrome and obesity.

The present disclosure provides a pharmaceutical composition comprising n-3 docosapentaenoic acid (DPA) or a derivative thereof in substantially pure form together with a pharmaceutically acceptable carrier or excipient. In one example, the composition does not raise the proportion of EPA in post-prandial triglycerides. In one example, the composition decreases post prandial chylomicronemia.

In one example, the n-3 DPA is provided in free fatty acid form. In one example, the n-3 DPA is provided in triglyceride form. In one example, the n-3 DPA is provided in ethyl ester form.

In one example, the n-3 DPA or derivative thereof comprises at least 10%, at least 20%, at least 30%, at least 40%, at least 50%, at least 60%, at least 70%, at least 80%, at least 90%, at least 95%, at least 96%, at least 97%, at least 98%, at least 99%, at least 99.5%, at least 99.8% by weight of the composition.

The present disclosure also provides a pharmaceutical composition comprising n-3 docosapentaenoic acid (DPA) or a derivative thereof for use in treating or preventing hypertriglyceridemia or treating or preventing post-prandial elevation in blood triglycerides in a subject in need thereof.

The present invention also provides a pharmaceutical composition comprising n-3 docosapentaenoic acid (DPA) or a derivative thereof for use in the treatment or prevention of a disorder associated with hypertriglyceridemia in a subject in need thereof.

In one example, the n-3 DPA is provided in free fatty acid form. In one example, the n-3 DPA is provided in triglyceride form. In one example, the n-3 DPA is provided in ethyl ester form.

The present disclosure provides for the use of purified n-3 docosapentaenoic acid (DPA) or derivative thereof, or a pharmaceutical composition comprising purified n-3 DPA or derivative thereof, for treating or preventing hypertriglyceridemia or treating or preventing post-prandial elevation in blood triglycerides in a subject in need thereof.

The present disclosure also provides for the use of purified n-3 docosapentaenoic acid (DPA) or a derivative thereof, or a pharmaceutical composition comprising purified n-3 DPA or derivative thereof, for the treatment or prevention of a disorder associated with hypertriglyceridemia in a subject in need thereof. In one example the subject is administered an effective amount of purified n-3 DPA or derivative thereof, or a pharmaceutical composition comprising n-3 DPA or a derivative thereof.

A significant finding determined by the present inventor was that administration of DPA almost completely eliminated the incorporation of fatty acids in chylomicrons which effect was not seen with the administration of EPA. In one example, administration of the composition does not raise the proportion of EPA in post-prandial triglycerides. In another example, the composition decreases post prandial chylomicronemia. In one example, a decrease in post-prandial chylomicronemia occurs within five hours of administration of purified n-3 DPA or derivative thereof or a composition comprising n-3 DPA or derivative thereof according to the present disclosure.

The present disclosure also provides use of purified n-3 DPA or a derivative thereof, or a pharmaceutical composition comprising n-3 DPA according to the present disclosure in medicine.

The present disclosure also provides for the use of purified n-3 DPA or a derivative thereof in the manufacture of a medicament for treating or preventing hypertriglyceridemia or treating or preventing post-prandial elevation in blood triglycerides in a subject in need thereof. In one example, the medicament according to the disclosure treats or prevents a disorder associated with hypertriglyceridemia in a subject.

The present disclosure also provides a method of reducing fasting triglycerides in a subject comprising administering to the subject an effective amount of purified n-3 docosapentaenoic acid (DPA) or a derivative thereof, or a pharmaceutical composition comprising n-3 DPA or a derivative thereof for a period effective to reduce fasting triglycerides in the subject. In one example, the method does not raise the proportion of EPA in post-prandial triglycerides. In one example, the method decreases post prandial chylomicronemia.

In one example, the subject being treated has a baseline fasting triglyceride level of at least about 200 mg/dl, at least about 300 mg/dl, at least about 400 mg/dl, at least about 500 mg/dl, at least about 600 mg/dl, at least about 700 mg/dl, at least about 800 mg/dl, at least about 900 mg/dl, at least about 1000 mg/dl, at least about 1100 mg/dl, at least about 1200 mg/dl, at least about 1300 mg/dl, at least about 1400 mg/dl, or at least about 1500 mg/dl. In one example, the subject being treated has a baseline triglyceride level, fed or fasting, from about 400 mg/dl to about 2500 mg/dl, about 450 mg/dl to about 2000 mg/dl or about 500 mg/dl to about 1500 mg/dl.

In one example, the subject has a fasting baseline triglyceride level of 500 mg/dl to about 2000 mg/dl.

It will be appreciated that the person skilled in the art will readily be able to determine whether a reduction of fasting triglycerides has occurred in a subject. In one example, a comparison is made between the fasting triglyceride levels measured prior to and following administration of n-3 docosapentaenoic acid (DPA) or a derivative thereof. The time period between the measurement of triglyceride levels will be at the discretion of the clinician, but may be at least 24 hours, a period of several days, weeks or months. The percentage reduction in fasting triglyceride levels may be at least 5%, at least 10%, at least 15%, at least 20%, at least 30%, at least 35%, at least 40%, at least 45%, at least 50% or greater.

The present disclosure also provides a method for treating or preventing hypertriglyceridemia or treating or preventing post-prandial elevation in blood triglycerides in a subject in need thereof, comprising administering to the subject an effective amount of purified n-3 DPA or derivative thereof, or a pharmaceutical composition comprising n-3 docosapentaenoic acid (DPA) or a derivative thereof.

The present disclosure also provides a method for the treatment or prevention of a disorder associated with hypertriglyceridemia in a subject in need thereof, comprising administering to the subject an effective amount of purified n-3 DPA or derivative thereof in purified form or a pharmaceutical composition comprising n-3 DPA or a derivative thereof.

In one example, according to any use, composition or method of the disclosure the n-3 DPA is provided in free fatty acid form. In one example, the n-3 DPA is provided in triglyceride form. In one example, the n-3 DPA is provided in ethyl ester form.

In one example, according to any use, composition or method of the disclosure, the treating causes a reduction in plasma triglycerides. In one example, according to any use, composition or method of the disclosure, the treating causes a reduction in plasma chylomicronemia.

In one example, according to any use, composition or method of the disclosure, blood triglycerides are plasma triglycerides, serum triglycerides or a chylomicron-rich fraction of the blood.

In one example, according to any use, composition or method of the disclosure, the purified n-3 DPA or derivative thereof or composition comprising n-3 DPA or derivative thereof reduced lipoprotein particle size compared with subjects not administered the purified n-3 DPA or composition thereof.

In one example, according to any use, composition or method of the disclosure, the purified n-3 DPA or derivative thereof or composition comprising n-3 DPA or derivative thereof comprises at least 10%, at least 20%, at least 30%, at least 40%, at least 50%, at least 60%, at least 70%, at least 80%, at least 90%, at least 95%, at least 96%, at least 97%, at least 98%, at least 99%, at least 99.5%, at least 99.8% by weight.

In one example, according to any use, composition or method of the disclosure, the purified n-3 DPA or derivative thereof or pharmaceutical composition comprising n-3 DPA or derivative thereof comprises not more than about 10%, not more than about 9%, not more than about 8%, not more than about 7%, not more than about 6%, not more than about 5%, not more than about 4%, not more than about 3%, not more than about 2%, not more than about 1%, not more than about 0.5% eicosapentaenoic acid (EPA) or docosahexaenoic acid (DHA) or a combination of EPA and DHA. In another example, the composition of the present disclosure contains substantially no DHA and/or EPA. In another example, the composition of the present disclosure contains no DHA and/or EPA or derivatives thereof. In another example, the n-3 DPA is bound to albumin.

In one example, according to any use, composition or method of the present disclosure, the disorder associated with hypertriglyceridemia is selected from (i) a cardiovascular-related disorder, (ii) rheumatoid arthritis, (iii) Raynaud Syndrome, (iv) lupus, (v) menstrual pain (vi) type II diabetes, (vii) obesity, (viii) Crohn's disease, (viv) osteoarthritis, (x) hypothyroidism, (xi) kidney disease and (xii) osteoporosis. In another example, the disorder is familial lipoprotein lipase deficiency (chylomicronemia syndrome).

In one example, according to any use or method of the present disclosure, the subject is on medication that causes plasma triglycerides to be raised above normal levels (i.e >150 mg/dl). In one example, the subject is taking medication selected from (i) tamoxifen, (ii) steroids, (iii) beta-blockers, (iv) diuretics. (v) estrogen, (vi) oral retinoids and (vii) birth control pills.

In one example, according to any use, composition or method of the present disclosure, the subject is an HIV subject who is on protease inhibitor medication.

In one example, according to any use, composition or method of the present disclosure, the subject is an alcoholic.

In one example, according to any use, composition or method of the present disclosure, the subject has familial lipoprotein lipase deficiency (chylomicronemia syndrome).

In one example, the subject according to any use, composition or method of the present disclosure has previously been treated with an agent selected from one or more of i) statins, ii) fibrates, iii) nicotinic acid, iv) Lovaza® (formulation comprising n-3 EPA ethyl ester and n-3 DHA ethyl ester) and v) Vascepa® (formulation comprising n-3 EPA) and has experienced an increase in, or no decrease in plasma triglyceride level. In one example, treatment with one or more of the above agents is discontinued and replaced by a use or method of the present disclosure.

In one example, the subject is not taking one or more of the following i) blood pressure medication, ii) anticoagulants, iii) diabetes medication, iv) asprin, v) cyclosporine and vi) topical corticosteroid for treatment of chronic psoriasis.

In one example, the present disclosure provides a method of reducing hypertriglyceridemia in a subject when treatment with a statin or niacin extended-release monotherapy is considered inadequate (Frederickson type IV hyperlipidemia).

In another example, the present disclosure provides a method of treating or preventing very high plasma triglyceride levels (e.g. Types IV and V hyperlipidemia) in a subject, comprising administering to the subject an effective amount of purified n-3 DPA or a derivative thereof, or a pharmaceutical composition comprising n-3 DPA or a derivative thereof as disclosed herein.

In one example, the subject being treated according to a use, composition or method of the present disclosure exhibits a fasting baseline absolute plasma level of total fatty acid not greater than about 250 nmol/ml, not greater than about 200 nmol/ml, not greater than about 150 nmol/ml, not greater than 100 nmol/ml or not greater than about 50 nmol/ml.

In another example, the subject exhibits a fasting baseline plasma, plasma, or red blood cell membrane n-3 DPA level not greater than about 70 μg/ml, not greater than about 60 μg/ml, not greater than about 50 μg/ml, not greater than about 40 μg/ml, not greater than about 30 μg/ml, or not greater than about 25 μg/ml.

In another example, the subject exhibits a fasting baseline plasma n-3 DPA level not greater than about 0.40% of total fatty acids in plasma.

In another example, the subject exhibits a fasting baseline erythrocyte n-3 DPA level not greater than about 1.8% of total fatty acids in erythrocytes.

In one example, the methods of the present disclosure comprise a step of measuring a subject's baseline lipid profile prior to initiating therapy. In another example, the methods of the present disclosure comprise the step of identifying a subject having one or more of the following:
(i) baseline non-HDL-C level of about 200 mg/dl to about 400 mg/dl, or at least about 210 mg/dl, or at least about 220 mg/dl, or at least about 230 mg/dl, or at least about 240 mg/dl, or at least about 250 mg/dl, or at least about 260 mg/dl, or at least about 270 mg/dl, or at least about 280 mg/dl, or at last about 290 mg/dl, or at least about 300 mg/dl;
(ii) baseline total cholesterol level of about 250 mg/dl to about 400 mg/dl, or at least about 260 mg/dl, or at least about 270 mg/dl, or at least about 280 mg/dl, or at least about 290 mg/dl, or at least about 300 mg/dl;
(iii) baseline VLDL-C level of about 140 mg/dl to about 200 mg/dl, or at least about 150 mg/dl, or at least about 160 mg/dl, or at least about 170 mg/dl, or at least about 180 mg/dl, or a least about 190 mg/dl;
(iv) baseline HDL-C level of about 10 to about 60 mg/dl, or not more than about 40 mg/dl, or not more than about 35 mg/dl, or not more than about 30 mg/dl, or not more than about 25 mg/dl, or not more than about 20 mg/dl, or not more than about 15 mg/dl; and/or
(v) baseline LDL-C level of about 50 to about 300 mg/dl, or not less than about 100 mg/dl, or not less than about 90 mg/dl, or not less than about 80 mg/dl, or not less than about 70 mg/dl, or not less than about 60 mg/dl, or not less than about 50 mg/dl.

In another example, the methods of the present disclosure comprise a step of measuring a subject's fasting apoB-48 levels. While not wishing to be bound by theory, serum apoB-48 levels have been found to correlate with plasma triglyceride concentrations but not with cholesterol levels (Sakai N et al (2003) Journal of Lipid Research vol 44:1256).

In one example, following treatment with purified n-3 DPA or a derivative thereof or a composition comprising n-3 DPA according to the disclosure, the subject exhibits one or more of the following outcomes:

(i) a reduction in serum or plasma triglyceride level of at least about 5%, at least about 10%, at least about 15%, at least about 20%, at least about 30%, at least about 35%, at least about 40%, at least about 45%, at least about 50%, at least about 55%, at least about 60%, or at least about 70% compared to baseline;

(ii) a less than 30% increase, less than 20% increase, less than 10% increase, less than 5% increase or no increase in non-HDL-C levels or a reduction in non-HDL-C levels of at least about 1%, at least about 3%, at least about 5%, at least about 10%, at least about 15%, at least about 20%, at least about 25%, at least about 30%, at least about 35%, at least about 40%, at least about 45%, at least about 50%, at least about 55% or at least about 75% compared to baseline;

(iii) substantially no change in HDL-C levels, no change in HDLC levels, or an increase in HDL-C levels of at least about 5%, at least about 10%, at least about 20%, at least about 25%, at least about 30%, at least about 35%, at least about 40%, at least about 45%, at least about 50%, at least about 55%, or at least about 75% compared to baseline;

(iv) less than 60% increase, less than 50% increase, less than 40% increase, less than 30% increase, less than 20% increase, less than 10% increase, less than 5% increase or no increase in LDL-C levels, or a reduction in LDL-C levels of at least about 5%, at least about 10%, at least about 15%, at least about 20%, at least about 25%, at least about 30%, at least about 35%, at least about 40%, at least about 45%, at least about 50%, at least about 55%, at least about 55%, or at least about 75% compared to baseline;

(v) a reduction in VLDL levels of at least about 5%, at least about 10%, at least about 15%, at least about 20%, at least about 25%, at least about 30%, at least about 35%, at least about 40%, at least about 45%, at least about 50% or at least about 100% compared to baseline.

In one example the subject exhibits a reduction in apolipoprotein B100 (apo B) level compared to baseline. In one example, the subject exhibits a reduction in apolipoprotein B48 level compared to baseline. In one example, the subject exhibits an increase in apolipoprotein A-I (apo A-I) level compared to baseline. In one example, the subject exhibits an increase in apo A-I/apo B100 ratio compared to baseline. In one example, the subject exhibits a reduction in lipoprotein (a) level compared to baseline. In one example, the subject exhibits a reduction in mean LDL particle number compared to baseline. In one example, the subject exhibits a reduction in oxidized LDL compared to baseline. In one example, the subject exhibits a reduction in phospholipase A2 compared to baseline. In one example, the subject exhibits a reduction in intracellular adhesion molecule-1 compared to baseline. In one example, the subject exhibits a reduction in plasminogen activator inhibitor-1 compared to baseline. In one example, the subject exhibits a reduction in total cholesterol compared to baseline. In one example, the subject exhibits a reduction in high sensitivity C-reactive protein (hsCRP) compared to baseline.

In one example according to any method or use according to the present disclosure, the subject fasts for up to 12 hours prior to consumption of purified n-3 DPA or derivative thereof or a composition comprising n-3 DPA or a derivative thereof according to the present disclosure. In one example, the subject fasts for 10 hours prior to consumption of purified n-3 DPA or derivative thereof or a composition comprising n-3 DPA or a derivative thereof according to the present disclosure.

In one example, the purified n-3 or derive thereof, or pharmaceutical composition comprising n-3 DPA or derivative thereof prevents elevation of post prandial triglyceride levels. In one example, postprandial triglycerides are prevented from being elevated for at least about 1-12 hours, at least about 2-10 hours, at least about 3-8 hours, or at least about 2-5 hours.

The present disclosure also provides a weight loss supplement comprising purified n-3 DPA or derivative thereof, or a composition comprising n-3 DPA or derivative thereof according to the present disclosure. In one example, the weight loss supplement is provided in an oral form which can admixed with a solid food or beverage. In one example, the weight loss supplement is provided as a capsule for oral ingestion. In one example, the weight loss supplement is used to treat an obese patient.

The present disclosure also provides for the use of purified n-3 DPA or a derivative thereof, or a composition comprising n-3 DPA or composition thereof according to the present disclosure in a weight loss supplement for treating or preventing obesity in a subject.

The present disclosure also provides a food additive comprising purified n-3 DPA or a derivative thereof, or a pharmaceutical composition comprising n-3 DPA or a derivative thereof. In one example, the food additive is added to a solid food. In one example, the food additive is added to liquid food. In one example, the food additive is added to animal feed.

The present disclosure also provides an animal feed comprising purified n-3 DPA or a derivative thereof, or a pharmaceutical composition comprising n-3 DPA or a derivative thereof according to the present disclosure.

The present disclosure also provides for the use of purified n-3 DPA or a derivative thereof as a food additive for treating or preventing a disorder associated with hypertriglyceridemia in a subject in need thereof. In one example, the n-3 DPA or derivative thereof is used as a food additive to a food selected from a functional food, nutrient-supplementing food, formula suitable for feeding infants or premature infants, baby foods, foods for expectant or nursing mothers, and geriatric foods. In one example, the n-3 DPA or derivative thereof is combined with carnitine and/or fibrates.

The present disclosure also provides for the use of purified n-3 DPA or a derivative thereof as a food additive for supplementing animal feed.

The present disclosure also provides use of purified n-3 DPA or a derivative thereof in a cosmetic formulation. In one example, the cosmetic formulation is a topical formulation. In one example, the topical formulation is a moisturising cream or lotion, bar soap, lipstick, shampoo or therapeutic skin preparation for dryness, eczema and psoriasis.

The present disclosure also provides a kit comprising purified n-3 DPA or a derivative thereof or a composition comprising n-3 DPA or derivative thereof as disclosed herein packaged together with instructions for use to treat hypertriglyceridemia or a disorder associated with hypertriglyceridemia in a subject in need thereof.

In one example, the purified n-3 DPA or a derivative thereof or a composition comprising n-3 DPA is administered to the subject from one to about four times per day.

In one example, the purified n-3 DPA or a derivative thereof or a composition comprising n-3 DPA is administered to the subject, prior to consumption of a meal, during consumption of a meal or immediately following consumption of a meal. In one example, the n-3 DPA or a derivative thereof or a composition comprising n-3 DPA is administered to the subject within one hour, within half and hour, or within 15 mins prior to consumption of a meal. In one example, the purified n-3 DPA or a derivative thereof or a composition comprising n-3 DPA is administered to the subject within 15 mins, within 30 mins or within 45 mins following consumption of a meal.

### DESCRIPTION OF THE FIGURES

FIG. 1 shows the triacylgylerol concentrations (mmol/l+/−standard deviation, n=10) in plasma (A) and chylomicron rich fraction (B) after the meals containing olive oil (open circles), or olive oil together with EPA (closed rectangles) or DPA (closed triangles). The incremental area under the chylomicron TAG curve after the DPA meal was significantly reduced when compared to the corresponding area after the olive oil meal (p=0.021) or the area after the EPA meal (p=0.034). In plasma, the difference between the TAG area after DPA and control meal tended to be significant (p=0.078). Significant differences (p<0.05) in individual time points between the olive oil breakfast and the DPA breakfast are marked by an asterisk.

FIG. 2 shows post prandial chylomicron triacyglycerol fatty acids (20:4n-6, 20:5n-3, 22:5n-3 and 22:6n-3) one to five hours after meals containing olive oil only (olive, white bars) or olive oil mixed with eicosapentaenoic acid (EPA, grey bars) or docosapentaenoic acid (DPA, black bars). Series of bars represent the times at which blood was drawn (1 to 5 hours) and an asterisk shows a significance between meal difference in the corresponding time point. Values are molar proportions (mean+/−standard deviation, n=10) of all chylomicron triacylglycerol fatty acids. Observed differences: EPA (20:5n3) 1-5 hr EPA meal and olive oil meal; 1-5 hr EPA meal and DPA meal; DPA (22:5n3) 2 h, 3 h (p=0.06), 4-5 hr DPA meal and olive oil meal; 3-5 h DPA meal and EPA meal; DHA (22:6n3) 2 h, 3 h DPA meal and olive oil meal; 5 h EPA meal to olive oil meal.

FIG. 3 shows post prandial chylomicron phospholipid fatty acids (20:4n-6, 20:5n-3, 22:5n-3 and 22:6n-3) one to five hours after meals containing olive oil only (olive, white bars) or olive oil mixed with eicosapentaenoic acid (EPA, grey bars) or docosapentaenoic acid (DPA, black bars). Series of bars represent the times at which blood was drawn (1 to 5 hours) and an asterisk shows a significance between meal difference in the corresponding time point. Values are molar proportions (mean+/−standard deviation, n=10) of all chylomicron phospholipid fatty acids. Observed differences: EPA (20:5n3) 2 h EPA meal and olive oil meal; 2 h EPA meal and DPA meal; DPA (22:5n3) 2 hr EPA meal and olive oil meal; DHA (22:6n3) 2 hr EPA meal and olive oil meal.

FIG. 4 shows PUFA containing triacylglycerols (acyl carbon number: number of double bonds) after the breakfasts containing olive oil (white bars), olive oil mixed with eicosapentaenoic acid (EPA, 20:5n-3, grey bars) and olive oil mixed with docosapentaenoic acid (DPA, 22:5n-3, black bars) at one, three and five hours, respectively. Semiquantitative values are expressed as molar percentages (mean+/−standard deviation, n=10) of all chylomicron TAGs. Most prevalent triacyglycerols based on the neutral loss experiments are marked above each group of bars.

### DETAILED DESCRIPTION

### General

The use of numerical values in the various quantitative values specified in this disclosure, unless expressly stated otherwise, are stated as approximations as though the minimum and maximum values within the stated ranges were both preceded by the word “about”. Also, the disclosure of ranges is intended as a continuous range including every value between the minimum and maximum values recited as well as any ranges that can be formed by such values.

The term “and/or”, e.g., “X and/or Y” shall be understood to mean either “X and Y” or “X or Y” and shall be taken to provide explicit support for both meanings or for either meaning. Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

Any example herein shall be taken to apply mutatis mutandis to any other example unless specifically stated otherwise.

### Selected Definitions

The term “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “chylomicron” as used herein refers to lipoprotein particles that consist of triglycerides (85-92%), phospholipids (6-12%), cholesterol (1-3%) and proteins (1-2%). Chylomicrons are one or the five major groups of lipoproteins (chylomicrons, VLDL, IDL, LDL, HDL) that enable fats and cholesterol to move within the bloodstream.

The term “DPA” as used herein is intended to refer to the omega-3 (w3 or n-3) and includes the natural form, being the triglyceride form, the free fatty acid form, the phospholipid form, as well as derivative forms prepared by chemical modification, conjugates, salts thereof or mixtures of any of the foregoing.

The term “derivative thereof” of DPA is understood to include the alkyl ester, ethyl ester, methyl ester, propyl ester, or butyl ester. In another example, the DPA is in the form of ethyl-DPA, lithium-DPA, mono-, di-, or triglyceride DPA or any other ester or salt of DPA, or the free acid form of DPA. DPA may also be in the form of a 2-substituted derivative or other derivative which slows down its rate of oxidation but does not otherwise change its biological action to any substantial degree.

The term “cardiovascular-related disease” as used herein refers to any disease or disorder of the heart or blood vessels (i.e. arteries and veins) and any symptom thereof. Non-limiting examples of cardiovascular-related disease and disorders include hypertriglyceridemia, hypercholesterolemia, mixed dyslipidemia, coronary heart disease, vascular disease, stroke, athersclerosis, arrhythmia, hypertension, myocardial infarction and other cardiovascular events.

The “subject” according to the present disclosure shall be taken to mean any subject, including a human or non-human subject. The non-human subject may include non-human primates, ungulate (bovines, porcines, ovines, caprines, equines, buffalo and bison), canine, feline, lagomorph (rabbits, hares and pikas), rodent (mouse, rat, guinea pig, hamster and gerbil), avian, and fish. In one example, the subject is a human. In one example, the subject consumes a traditional Western diet.

The term “Western diet” as used herein refers generally to a typical diet consisting of, by percentage of total calories, about 45% to about 50% carbohydrate, about 35% to about 40% fat and about 10% to about 15% protein. A Western diet may alternately or additionally be characterized by relatively high intakes of red and processed meats, sweets, refined grains and desserts, for example more than 50%, more than 60% or more or 70% of total calories from these sources.

The term “triglyceride” as used herein is intended to refer to an ester composed of a glycerol bound to three fatty acids. Triglycerides can be divided into saturated and unsaturated compounds. Saturated compounds are saturated with hydrogen, meaning all available places where hydrogen atoms could be bonded to carbon atoms are occupied. Unsaturated compounds have double bonds between carbon atoms, reducing the number of places where hydrogen atoms can bond to carbon atoms. Saturated compounds have single bonds between carbon atoms and the other bond is bound to hydrogen atoms. Unsaturated fats have a higher melting point and are more likely to be solid. Triglycerides cannot pass through cell membranes freely. Lipoprotein lipases must break down triglycerides into free fatty acids and glycerol. The free fatty acids can be then taken up by cells via the fatty acid transporter. Triglycerides are major components of very low density lipoproteins and chylomicrons and play an important role in metabolism as energy sources and transporters of dietary fat.

The term “hypertriglyceridemia” as used herein is intended to refer to elevation in plasma or serum triglyceride levels above fasting levels and typically refers to high blood levels of triglycerides. High triglyceride levels are typically in the range of about 200 to about 499 mg/dl. Very high triglyceride levels are typically >500 mg/dl. Baseline triglycerides are typically measured when the subject is in a fasting state, that is, the subject has fasted for a period of between 8 and 12 hours.

The term “fatty acid” as used herein refers to a molecule that is derived from a triglyceride or phospholipid and is comprised of a carboxylic acid with a long aliphatic tail (chain) which is either saturated or unsaturated. When not attached to other molecules, they are known as “free” fatty acids. Most naturally occurring fatty acids have a chain of an even number of carbon atoms, from 4 to 28. Short chain fatty acids (SOFA) are fatty acids with aliphatic tails of fewer than six carbons. Medium chain fatty acids (MCFA) are fatty acids with aliphatic tails of 6-12 carbons which can form medium chain triglycerides. Long chain fatty acids (LCFA) are fatty acids with aliphatic tails 13 to 21 carbons. Very long chain fatty acids (VLCFA) are fatty acids with aliphatic tails longer than 22 carbons.

The term “polyunsaturated fatty acids” or PUFAs as used herein are intended to refer to fatty acids that contain more than one double bond in their backbone. Unsaturated refers to the fact that the molecules contain less than the maximum amount of hydrogen. Polyunsaturated fatty acids may be divided into omega 3 and omega 6 type fatty acids. Omega 3 fatty acids have a double bond that is three carbons away from the methyl carbon. Examples of omega 3 polyunsaturated fatty acids include hexadecatrienoic acid (16:3 (n-3)), alpha-linolenic acid (18:3 (n-3)), stearidonic acid (18:4 (n-3)), eicosatrienoic acid (20:3 (n-3)), eicosatetraenoic acid (20:4 (n-3)), eicosapentaenoic acid (20:5 (n-3)), heneicosapentaenoic acid (21:5 (n-3)), docasapentaenoic acid (22:5 (n-3)), docosahexaenoic acid (22:6 (n-3)), tetracosapentaenoic acid (24:5 (n-3)), tetracosahexaenoic acid (24:6 (n-3)).

As used herein, the term “effective amount” shall be taken to mean a sufficient quantity of DPA or derivative or conjugate thereof to reduce fasting triglycerides in the subject having a fasting baseline triglyceride level of 500 mg/dl to about 2000 mg/dl and/or sufficient to reduce or alleviate a cardiovascular disease or disorder in a subject. The skilled artisan will be aware that such an amount will vary depending on, for example, the particular subject and/or the type or severity or level of disease. Accordingly, this term is not to be construed to limit the composition of the disclosure to a specific quantity, e.g., weight or amount of DPA and/or derivative(s), rather the present disclosure encompasses any amount of DPA and/or derivative(s) sufficient to achieve the stated result in a subject. In one example, an “effective amount” is a therapeutically effective amount”.

As used herein, the term “therapeutically effective amount” shall be taken to mean a sufficient quantity of DPA to reduce, inhibit or prevent one or more symptoms of a clinical disorder associated with elevated triglyceride levels to a level that is below that observed and accepted as clinically diagnostic or clinically characteristic of that disorder. The term also means that the substance in question does not produce unacceptable toxicity to the subject or interaction with other components in the composition. The skilled artisan will be aware that such an amount will vary depending on, for example, the particular subject and/or the type or severity or level of disorder. Accordingly, this term is not to be construed to limit the composition of the disclosure to a specific quantity, e.g., weight or amount of DPA and/or derivative(s), rather the present disclosure encompasses any amount of DPA and/or derivative(s) sufficient to achieve the stated result in a subject.

As used herein, the terms “treating”, “treat” or “treatment” include administering a therapeutically effective amount of n-3 DPA described herein sufficient to reduce or eliminate at least one symptom of a specified disorder. In one example, the treatment involves administering a therapeutically effective amount of n-3 DPA to reduce plasma triglyceride levels. In one example, the reduction is measured over a specific time period against a baseline level of fasting plasma triglycerides. In one example, the reduction in plasma triglyceride levels is at least about 5%, at least about 10%, at least about 15%, at least about 20%, at least about 25%, at least about 30%, at least about 35%, at least about 40%, at least about 45%, at least about 50%, at least about 60%, at least about 70%, at least about 80% or at least about 90% compared to baseline. In one example, treatment also refers to prophylactic treatment.

As used herein, the terms “preventing”, “prevent” or “prevention” include administering a therapeutically effective amount of n-3 DPA described herein sufficient to stop or hinder the development of at least one symptom of a specified disorder. In one example, the administration of n-3 DPA or derivative thereof prevents post prandial elevation of plasma triglycerides.

The term “substantially purified” is understood to mean that the n-3 DPA or derivative thereof is substantially free of cellular material or other contaminating proteins from the source from which the DPA is derived. In one example, the n-3 DPA or derivative thereof comprises at least 10%, at least 20%, at least 30%, at least 40%, at least 50%, at least 60%, at least 70%, at least 80%, at least 90%, at least 95%, at least 96%, at least 97%, at least 98%, at least 99%, at least 99.5%, at least 99.8% by weight. The term is also understood to mean that a composition comprising n-3 DPA or derivative thereof comprises not more than about 10%, not more than about 9%, not more than about 8%, not more than about 7%, not more than about 6%, not more than about 5%, not more than about 4%, not more than about 3%, not more than about 2%, not more than about 1%, not more than about 0.5% EPA or DHA or a combination of EPA and DHA.

### Measurement of Triglycerides and Cholesterol Lipoproteins

Measurement of lipid parameters may be in accordance with any clinically acceptable methodology. For example, triglycerides, total cholesterol, HDL-C and fasting blood sugar can be sampled from plasma and analysed using standard photometry techniques or by gas chromatography according to art known methods. LDL-C and VLDL-C can be calculated or determined using plasma lipoprotein fractionation by preparative ultracentrifugation and subsequent quantitative analysis by refractometry or by analytic ultracentrifugation methodology. Apo A1, Apo B and hsCRP can be determined from plasma using standard nephelometry techniques. Lipoprotein (a) can be determined from plasma using standard turbidimetric immunoassay techniques. Phospholipase A2 can be determined from EDTA plasma or serum using enzymatic immunoseparation techniques. Oxidised LDL and intracellular adhesion molecule-1 can be determined from plasma using standard enzyme immunoassay techniques. These techniques are described in detail in standard textbooks, for example Tietz Fundamentals of Clinical Chemistry, 6^{th} Ed. (Burtis, Ashwood and Borter Eds.), WB Saunders Company.

Docosapentaenoic Acid (22:5n-3) DPA

n-3 DPA in both plasma and erythrocytes has been shown to have little correlation with dietary fish or long chain n-3 fatty acid intake (Jing Qi Sun et al (2008) Am J Clin Nutr 88:216-23).

In vivo, DPA is formed by chain elongation of EPA by the action of fatty acid elongases 2 and 5, while the conversion of DPA to DHA requires an elongation to 24:5n-3 and desaturation to 24:6n-3 before peroxisomal beta-oxidation to yield DHA. As recently reviewed, ALA supplementation generally leads to an increase in plasma EPA and DPA, but has little or no effect on DHA levels (Brenna et al., (2009) Prostaglandins Leukot Essent Fatty Acids 80:85-91).

There is another isomer of DPA which is an n-6 fatty acid. The n-6 DPA content is low in most mammalian tissues, except testes tissue. In fish and fish oils, the n-3 isomer of DPA is substantially higher than the n-6 isomer. The physiological behaviour of n-3 and n-6 DPA differ profoundly despite only differing in the position of two double bonds in the acyl chain.

n-3 DPA has not been extensively studied because of the limited availability of the pure compound. In vitro n-3 DPA is retro-converted back to EPA, however it does not appear to be readily metabolized to DHA. In vivo studies have shown limited conversion of n-3 DPA to DHA, mainly in liver, but in addition, retro-conversion to EPA is evident in a number of tissues.

### Utility of n-3 DPA

The present findings suggest an important role for purified n-3 DPA in lowering plasma triglyceride levels in subjects in need thereof. Such subjects may be those at risk of cardiovascular disease caused either through diet or hereditary mechanisms.

The fact that triglyceride levels remained close to fasting levels following consumption of n-3 DPA alone suggest that n-3 DPA alone may provide a potent alternative to EPA containing compounds on the market and in particular, may provide a useful weight loss supplement for subjects wishing to control their weight or lose weight. Thus, the present findings also suggest a role for purified n-3 DPA in obesity treatment. New tools for fighting the growing prevalence of obesity worldwide are needed. Currently orlistat, a lipase inhibitor is the only available long-term treatment for obesity. In the past years, numerous drugs have been approved for the treatment of obesity; however most of them like amphetamine, rimonabant and sibutramine have been withdrawn from the market because of their adverse effects.

Lovaza (in the USA) and Omacor® (in Europe) sold by GlaxoSmithKline has been approved by the US Food and Drug Administration to lower very high triglyceride levels. Each 1 g capsule contains at least 900 mg of the ethyl esters of omega-3 fatty acids sourced from fish oils. These are predominantly a combination of ethyl esters of eicosapentaenoic acid (EPA-approximately 465 mg, about 50% EPA) and docosahexaenoic acid (DHA-approximately 375 mg, about 40%) with the reminder constituting other fatty acids from fish oils. Lovaza also contains the inactive ingredients α-tocopherol, gelatin, glycerol and purified water (see http://us.gsk.com/products/assets/us_lovaza.pdf for prescribing information). Lovaza has been demonstrated to reduce triglycerides in patients with high or very high triglycerides and has been demonstrated to reduce VLDL-cholesterol and non-HDL cholesterol, and increase HDL-cholesterol. However. Lovaza can raise LDL-cholesterol up to 45% and elevate alanine transaminase levels which can lead to liver damage.

In July 2012, Amarin Corporation's drug, Vascepa received FDA approval. The drug has been approved for treatment of high triglycerides and well as very high triglycerides. Each capsule of Vascepa contains the ethyl ester of eisosapentaenoic acid (EPA).

The potency of purified n-3 DPA observed in these studies suggest that purified n-3 DPA may provide a viable and possibly superior alternative to currently marketed triglyceride lowering drugs.

### Purification of DPA

Omega-3 fatty acids are found in nature in the triglyceride form (a glycerol with three fatty acids attached) and the phospholipid form (glycerol with two fatty acids and a base such as choline). These are the main lipids that would have been ingested by human ancestors during evolution. Fish oil and seal oil have been found to contain n-3 DPA as well as n-3 EPA and n-3 DHA. However given the low proportion of n-3 DPA relative to the other fatty acids, it has been extremely difficult to isolate n-3 DPA in pure form for analysis in subjects.

n-3 DPA may be obtained from fats and oils of marine animals such as mackerel, sardines, herring, cod, tuna, saury etc and animal marine plankton or seal fat or oil, however its concentration is very low in these sources. Furthermore, there are significant ethical issues associated with obtaining n-3 DPA from seal fat or oil in sufficient quantities for therapeutic utility. n-3 DPA can also be produced synthetically by standard techniques from n-3 EPA by addition of 2 carbon atoms to n-3 EPA followed by chromatographic purification using HPLC and blending with anti-oxidant.

Various processes for the production of n-3 EPA have been described. For example, U.S. Pat. No. 5,840,944 describes a method of producing pure EPA or their esters whereby a mixture of fatty acids or their esters produced from natural oils and fats is precision distilled under a high vacuum using a plurality of distillation columns to derive a fraction containing EPA which is then subjected to a reversed-phase partition type column chromatography. Other examples of EPA purification are described in for example, U.S. Pat. No. 4,331,695, U.S. Pat. No. 4,377,526, U.S. Pat. No. 4,615,839, U.S. Pat. No. 4,792,418, U.S. Pat. No. 5,006,281, U.S. Pat. No. 5,518,918, U.S. Pat. No. 5,130,061, U.S. Pat. No. 6,451,567, U.S. Pat. No. 6,800,299, U.S. 68/446,942, US 2005/0129739, US 2011/0098356, U.S. Pat. No. 7,119,118 Abu-Nasr et al (1954) J. Am. Oil Chemists Soc 31:41-45, Teshima et al (1978) in Bulletin of the Japanese Society of Scientific Fisheries 44(8):927, and Belarbi El Hassan et al (2000) Enzyme and Microbial Technology 26:516-529).

By way of non-limiting example, one method for preparing EPA is described in the following paragraphs. The oil from which EPA is obtained is preferably as fresh as possible so as to avoid any substantial degradation of the fatty acids. Preferably, the source fish are obtained from as cold an environment as possible. The optimal enzymatic activity for the enzyme Δ5-desaturase, which catalyzes the conversion of eicosatetraenoic acid to EPA, occurs at 9° C. Thus, fish from cold environments are higher in EPA than are fish from warmer waters. Even greater yields of EPA can be obtained if the fish are raised in a controlled environment. If the fish are fed a diet rich in α-linolenic acid and maintained in salt water at 9° C., optimum amounts of EPA will be produced.

The natural fat or oil containing EPA is subjected to saponification or alcoholysis in order to convert the triglycerides to free fatty acids or esters of fatty acids. The method selected, however should be one which avoids high temperatures and strongly basic conditions as these can lead to peroxidation and cis-trans conversion. In one example, the method of hydrolysis is enzymatic hydrolysis using the enzyme lipase at a temperature of about 35 to 40° C. and a pH of about 6-7. The lipase should be activated by traces of cysteine or ascorbic acid as is conventional. An alternative method for hydrolyzing the natural fats and oils is by partially hydrolyzing these fats and oils with lipase or a base. A base such as potassium hydroxide or sodium hydroxide can also be used to partially hydrolyse the natural fats or oils. The source of oil is treated with the base for about 15-20 minutes to partially hydrolyze the triglycerides.

After the hydrolysis step, unsaponified materials are removed with an organic nonpolar solvent such as methylene chloride, petroleum ether, ethyl ether etc. The organic solvent removes cholesterol, PCBs and other non-saponified materials, including vitamins A and D and hydrocarbons. This procedure is repeated several times until the desired purity is reached.

Free fatty acids can be formed from the sodium or potassium salt by acidifying the aqueous phase. Any acid can be used for this step, although pharmaceutically acceptable such as acetic acid is preferred. This acidification will cause the free fatty acids to separate into a separate organic phase. The aqueous phase is then discarded. Adding a small amount of a salt such as sodium chloride will enhance the separation. The organic phase, containing free fatty acids is then dissolved in acetone and refrigerated at about −20° C. overnight. The saturated fatty acids solidify and can be removed by filtering.

Omega-3 fatty acids can be obtained from the acetone solution by adding a mixture of a base such as sodium hydroxide and ethanol. The mixture is then left overnight under refrigeration at about −20° C. The acetone is then evaporated. This process can be repeated several times to reduce the amount of water. The free fatty acids can be protected from oxidation by adding a conventional, pharmaceutically acceptable or food-grade antioxidant, such as ascorbyl palmitate or γ-tocopherol.

Individual DHA and EPA omega-3 fatty acids can be separated from each other by forming salts of the acids which have different solubilities. For example, the magnesium salts of the acids have different solubilities in acetone. The solution is left overnight under refrigeration at about −20° C. The EPA salt, which is less soluble in acetone than the DHA salt, precipitates as white flakes. The white flakes are filtered out and reconstituted from the salt by acidifying. The DHA salt remains in solution, and the DHA can be obtained by acidifying the solution and recovering the free DHA by conventional means. Pure ω-3 fatty acids can be obtained based upon the difference in solubility in acetone of the magnesium or other group II metal salts that are soluble in acetone salts of the fatty acids. While the exemplified process has been described with respect to the use of acetone as the organic solvent from which the fatty acid EPA salt is precipitated upon cooling, it should be understood that an other organic solvents can be used for this purpose.

The precise temperatures to which the solutions are cooled to separate EPA from DHA, and the precise amounts of volume reduction, will differ depending upon the particular EPA and DHA salts and the particular solvent. These parameters can be empirically determined by those skilled in the art without undue experimentation. The solution may be cooled to a temperature slightly below that at which precipitation begins, and maintained at that temperature until precipitation is completed.

Alternatively, the free fatty acid of EPA can be separated from the other fatty acids by use of chromatography (e.g. HPLC). Purification may also be carried out after conversion of the fatty acids to esters of lower alcohols. Esterification can be carried out using known conditions, for example treatment by reagents such as 5-10% HCL-anhydrous ethanol solution, 10-50% BF₃-ethanol solution, for 1-24 hours at room temperature. Column chromatography, low temperature crystallization, urea addition, liquid-liquid counter current distribution chromatography and such may be used alone or in combination to isolate the EPA ethyl ester from the mixture.

In order to obtain free EPA from the purified EPA ethyl ester, the ester can be hydrolysed by alkali and then extracted with organic solvents such as ether, ethyl acetate and such. The obtained free EPA can then be used to derive DPA.

Production of DPA from EPA requires an elongation reaction. Such techniques will be familiar to persons skilled in the art but see for example U.S. Pat. No. 7,968,692, and U.S. Pat. No. 8,071,341. The DPA is then purified by chromatography, for example HPLC.

Purified n-3 DPA (e.g. Maxomega DPA 97 FFA) can be obtained from a commercial source e.g. Equateq (now BASF). Maxomega DPA 97 FFA contains 97% by weight DPA in the free fatty acid form and is a synthetic fatty acid produced from a natural marine EPA ethyl ester concentrate (EPA 98 FFA). EPA (Maxomega EPA 98 FFA) is a purified product derived from fish oil. Fish oil is purified by standard purification and refining techniques, is subjected to trans-esterification, concentrated by distillation and chromatography, converted to fatty acid form by hydrolysis, purified and blended with anti-oxidant. DPA 97 FFA is produced from EPA 98 FFA by standard synthetic procedures before being purified and blended with antioxidant.

### Compositions

Any biologically acceptable dosage forms, and combinations thereof may be contemplated by the present disclosure. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, cereals, cereal coatings, foods, nutritive foods, functional foods and combinations thereof. The preparations of the above dosage forms are well known to persons of ordinary skill in the art.

Pharmaceutical compositions useful in accordance with the methods of the present disclosure are orally deliverable. The terms “orally deliverable” or “oral administration” herein include any form of delivery of a therapeutic agent (e.g. n-3 DPA or a derivative thereof) or a composition thereof to a subject, wherein the agent or composition is placed in the mouth of the subject, whether or not the agent or composition is swallowed. Thus “oral administration” includes buccal and sublingual as well as oesophageal administration. In one example, the purified n-3 DPA or derivative thereof is present in a capsule, for example a soft gelatin capsule.

The pharmaceutical compositions according to the present disclosure are not limited with regard to their mode of use. Representative modes of use include foods, food additives, medicaments, weight supplements, additives for medicaments, and feedstuffs.

Examples of food compositions, besides general foods, are functional foods, nutrient-supplementing foods, formula suitable for feeding infants, baby foods, foods for expectant or nursing mothers, and geriatric foods. The composition may be added upon cooking such as soup, food to which oils and fat are used as heating medium such as doughnuts, oils and fat food such as butter, processed food to which oils and fat are added during processing such as cookies or food to which oils and fat are sprayed or applied upon completion of processing such as hard biscuits.

Furthermore the compositions of the present disclosure can be added to foods or drinks which do not normally contain oils or fat.

The definition of food also includes functional food. Functional foods and medicaments may be provided in processed form such enteral agent for promoting nutrition, powder, granule, troche, internal solution, suspension, emulsion, syrup, capsule and such.

The compositions according to the present disclosure can be formulated as one or more dosage units. The term “dose unit” and “dosage unit” herein refer to a portion of a composition that contains an amount of a therapeutic agent suitable for single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

In one example, a composition of the present disclosure is administered to a subject over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks, or about 1 week.

In one example the compositions of the present disclosure comprise one or more antioxidants (e.g. tocopherol) or other impurities in an amount of not more than about 0.5%, or not more than 0.05%. In another example, the compositions of the present disclosure comprise about 0.05% to about 0.4% tocopherol, or about 0.4% tocopherol, or about 0.2% by weight tocopherol.

In one example, the compositions of the present disclosure include one or more additional excipients including, but not limited to gelatin, glycerol, polyol, sorbitol and water.

In one example, the n-3 DPA or derivative thereof is present in the composition in an amount of about 50 mg to about 5000 mg, about 75 mg to about 2500 mg, or about 100 mg to about 1000 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In one example the compositions of the present disclosure comprise about 300 mg to about 1 g of the composition in a capsule. In one example, the dosage form is a gel or liquid capsule and is packaged in blister packages of about 1 to about 20 capsules per sheet.

In one example, a composition of the present disclosure is administered to a subject once or twice per day. In another example, the composition is administered to a subject as 1, 2, 3, or 4 capsules daily.

The composition may be administered to a subject in need thereof immediately before a meal, during consumption of the meal or shortly following the meal.

In another example, the composition of the present disclosure is formulated for topical application, for example in a cosmetic. Topical products that may incorporate n-3 DPA according to the present disclosure include moisturizing creams and lotions, bar soaps, lipsticks, shampoos and therapeutic skin preparations for dryness, eczema and psoriasis.

### Kits

The present disclosure also provides kits comprising purified n-3 DPA or a composition according to the present disclosure together with instructions for use in the present treatment methods. Such kits will generally contain a dosage form packaged in blister packages of about 1 to 20 capsules per sheet or in a suitable container means of about 20 to 100, or about 20 to 50 individual capsules. The kit will typically include written prescribing information.

The present invention is described further in the following non-limiting examples.

### Examples

### Methods

### Volunteer Subjects

Ten healthy normal weight females between the age of 20 to 30 of took part in a randomized cross over study with three different breakfast meals. The subjects had a BMI between 20 to 25 kg/m² and their habitual total consumption of omega-3 PUFA was not more than 500 mg per day as assessed from a PUFA food frequency questionnaire (Sullivan et al., (2006) Lipids 41:845-850; Swierk et al., (2011) Nutr 6:641-646). The baseline values for EPA and DHA proportions in erythrocytes were 1.0±0.1 and 6.7±0.6% (mean±standard error of the mean). Subjects with any form of cardiovascular disease based on self reported medical status and family history were excluded from the study. All subjects provided written informed consent. Ethics approval was obtained from the Deakin University Human Research Ethics Committee (EC2011-023).

### Study Procedure

This was a postprandial study where fasting blood was taken, followed by the consumption of a breakfast containing placebo (olive oil) or active oils (EPA or DPA) and then hourly post prandial blood samples were taken up to 5-hours. On the subsequent 6 days, the subjects continued to take the placebo or active oil and then after a fasting blood sample was taken on day 7, the subjects had a two week ‘washout’ period following the 7 day period.

The night before the study the participants consumed a standardized dinner meal (containing pasta (dry 200 g), tomato stir-through sauce (70 g) and a packet pudding and were given instructions to fast overnight for 10 hours after the dinner.

The study breakfast consisted of 180 grams of instant mashed potato (Continental Deb™, Unilever, Australasia) mixed with 70 ml boiled water and 20 grams of oil. In each of the three meals 18 grams of lipids consisted of olive oil (La Espanola Pure Olive Oil, Seville, Spain). The DPA breakfast included 2 g of DPA (Equateq Ltd, Breasclete, Callanish, Scotland), the EPA breakfast 2 g of EPA (Equateq Ltd, Breasclete, Callanish, Scotland) and the control (olive oil) meal an additional 2 g of olive oil. EPA and DPA were included in the olive oil as free fatty acids. The subjects could use salt, pepper or chicken flavoured salt with the meal and were provided with water throughout the study period ad libitum. Subjects consumed the study meal within 15 minutes.

After the DPA meal, there were two cases of diarrhea and one case of upset stomach but no diarrhea. One case of diarrhea was reported after the EPA meal, and there were no complaints after the olive oil meal. All complaints occurred 2 to 3 hours after the breakfast.

During the three week period of the cross-over trial, subjects were requested to refrain from consuming products rich in long chain omega-3 PUFA products including fish, red meat and omega-3 fortified products (<2 marine and/or 2 red meat meals/week and <2 omega-3 fortified products/week).

### Isolation of Plasma, Chylomicrons and Chylomicron Lipids

Venous blood was drawn at the fasting state and hourly post prandial for one to five hours. EDTA blood samples were immediately centrifuged for fifteen minutes at 591×g to isolate the plasma.

A chylomicron-rich fraction (Svedberg flotation unit (Sf) >400), later abbreviated to “chylomicrons”, was isolated from plasma by ultracentrifugation using a Beckman ultra centrifuge and TLA 100.4 rotor (Beckman instruments, Palo Alto, Calif., USA) as previously described (Ågren et al., 2006). Briefly 1.8 ml of EDTA plasma was overlaid with saline solution (density=1.006 kg/I) in ultracentrifuge tubes and centrifuged at 35,000×g for 30 min at 23° C. The top 1 millilitre was aspirated to remove the chylomicron-rich fraction. All samples were frozen at −80° C. prior analysis.

### TAG-Concentration Analysis

TAG concentrations in plasma and the isolated chylomicrons were measured on a Roche Cobas Integra 400 plus autoanalyser (Roche, Lavel, Quebec, Canada) by enzymatic colorimetric method using commercially available kits (TRIGL) as per the manufacturer's instructions (Roche, Lavel, Quebec, Canada).

Fatty acid analysis

An internal standard mixture of triheptadecanoin (Sigma-Aldrich, St. Louis, Mo., USA), dinonadecanoylphosphatidylcholine (Sigma-Aldrich, St. Louis, Mo., USA) and cholesterylpentadecanoate (Nu-Chek Prep. Inc., Elysian, Minn., USA) was added to the isolated chylomicrons. Then 1.5 ml methanol, 3 ml chloroform and 0.8 ml 0.88% KCl in water were added and the blend was thoroughly vortexed after each addition. The tubes were centrifuged 2000×g for 3 minutes to separate the layers, and the chloroform rich layer was removed and evaporated to dryness (Folch et al., (1957) J Biol Chem 226:497-509). TAGs and phospholipids were isolated from the extracted lipid mixture with solid phase extraction based on silica columns (Hamilton and Comai, (1988) Lipids 23:1146-1149).

Fatty acid methyl esters (FAME) were prepared with a sodium methoxide method. In short, the lipids were suspended to 1 ml dry diethylether. 25 μl methylacetate and 25 μl sodium methoxide were added and the blend was incubated for 5 minutes while shaken at times. The reaction was stopped with 6 μl acetic acid. The tubes were centrifuged 2000×g for 5 minutes, after which the supernatant was removed and gently evaporated to dryness. The resulting FAME were transferred to 100 μl inserts in hexane (Christie (1982) J Lipid Res 23:1072-1075). The FAME were analysed with gas chromatography (Shimadzu GC-2010 equipped with AOC-20i auto injector, flame ionization detector (Shimadzu corporation, Kyoto, Japan) and wall coated open tubular column DB-23 (60 m×0.25 mm i.d., liquid film 0.25 μm, Agilent technologies, J.W. Scientific, Santa Clara, Calif., USA). Splitless/split injection was used and the split was opened after 1 min. Supelco 37 Component FAME Mix (Supelco, St. Louis, Mo., USA), 68D (Nu-Check-Prep, Elysian, Minn., USA) and GLC-490 (Nu-Check-Prep, Elysian, Minn., USA) were used as external standards.

### Lipidomics

Lipidomic analysis of the one, three and five hour chylomicron samples was performed by liquid chromatography, electrospray ionisation-tandem mass spectrometry using an Applied Biosystems 4000 QTRAP mass spectrometer running Analyst 1.5 software. Liquid chromatography was performed on a Zorbax C18, 1.8 μm, 50×2.1 mm column (Agilent technologies, Santa Clara Calif., USA). The lipids of the chylomicrons were extracted with chloroform:methanol (2:1, 20 volumes), mixed, sonicated (30 mins) and allowed to stand for 20 mins. Samples were centrifuged (16 000×g, 10 min) and the supernatant transferred to a 96 well PPE plate and dried until a stream of nitrogen at 40° C. Immediately before analaysi, samples were resuspended in water saturated butanol and methanol containing 10 mM ammonium formate (NH₄COOH). The mobile phase was tetrahydrofuran:methanol:water in a 30:20:50 ratio (A) and 75:20:5 (B) both containing 10 mM NH4COOH. TAG were separated with an isocratic flow (100 μL/min) of 85% mobile phase B. Phospholipids and cholesteryl esters were separated by a gradient from 0% B and 100% A to 100% B and 0% A over 8 minutes then held at 100% B for 2 mins before equilibrating to starting conditions. Quantification of individual TAG species was performed using scheduled multiple-reaction monitoring (MRM) in the positive ion mode (Murphy et al., (2007) Anal Biochem 366:59-70). Lipid concentrations (pmol/mL) were calculated by relating the peak area of each species to the peak area of the internal standard of triheptadecanoin (Sigma Aldrich, St Louis Mo., USA) for TAGs, cholestyl ester-18:0D6 (CDN isotopes, Quebec, Canada) for cholesteryl esters, phosphatidyl choline-13:0/13:0 (Avanti Polar Lipids, Alabaster Ala., USA) for phosphatidyl cholines and phosphatidyl ethanolamine-17:0/17:0 (Avanti Polar Lipids, Alabaster Ala., USA) for ethanolamines and phosphatidyl inositols (using Multiquant 1.2 software). As no standards were available for each TAG species, no adjustment was made for different response factors and the relative proportions of different species should be taken as semi-quantitative.

TAGs that were likely to contain arachidonic acid, EPA, DPA or DHA were selected for further neutral loss experiments. Each molecular species selected was screened for the neutral loss of 16:0, 16:1, 18:1, 18:2, 18:3, 20:4, 20:5, 22:5 and 22:6. The most likely TAG fatty acid combinations were estimated from the results.

### Statistical Analyses

Normal distribution of the data was tested with the Shapiro-Wilk test. Depending on the normality of the data, paired samples t-test or Wilcoxon matched-pairs signed ranks test, was used to compare the measured responses to control. ANOVA for repeated measurements (GLM) was used for multiple comparisons. Paired samples t-test or Wilcoxon matched-pairs signed ranks test with Bonferroni correction was used for post hoc comparisons. Statistical significance was indicated by p<0.05. Statistical analyses were performed with SPSS 18.0 software (SPSS Inc, Chicago, Ill., USA).

### Results

### Example 1

### Triacylglycerol Concentration (mmol/l)

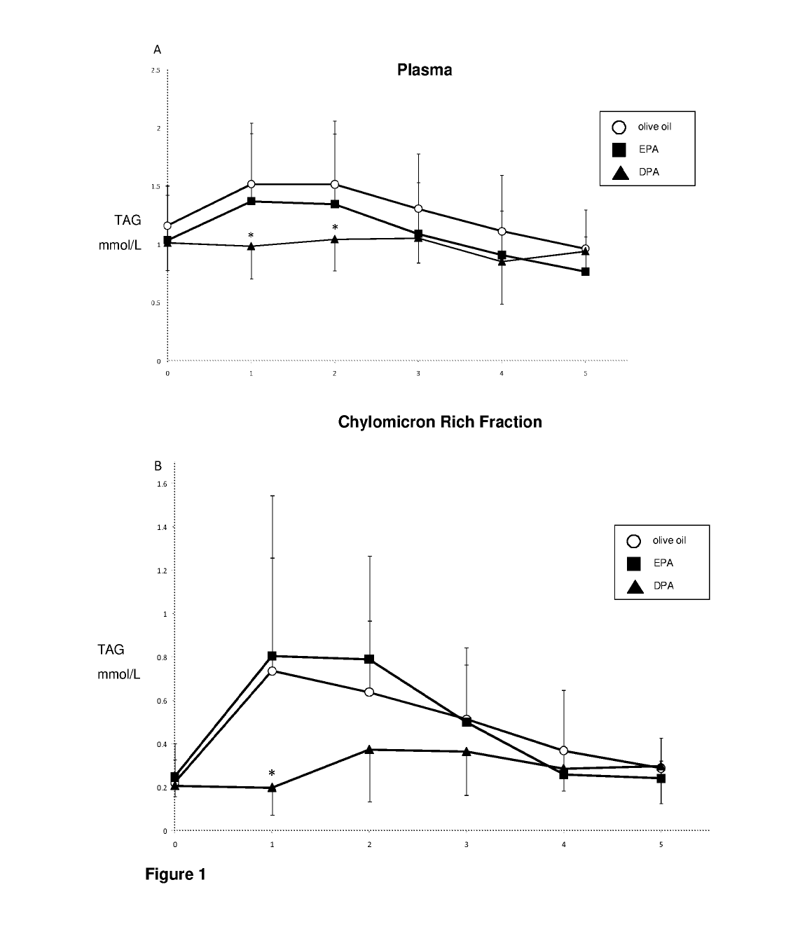

Chylomicron TAGs remained at almost fasting level after the DPA breakfast (FIG. 1). The incremental area under the chylomicron TAG curve after the DPA meal was significantly reduced when compared to the corresponding area after the olive oil meal (p=0.021) or the area after the EPA meal (p=0.034). In plasma, the difference between the TAG areas after DPA and control meal tended to be significant (p=0.078). Of the individual time points. the TAG concentration was smaller after the DPA breakfast at one and two hours (p=0.024 and p=0.014 respectively for plasma and p=0.017 and p=0.068 respectively for chylomicrons) compared to the control meal (FIG. 1).

### Example 2

### Chylomicron TAG Polyunsaturated Fatty Acids

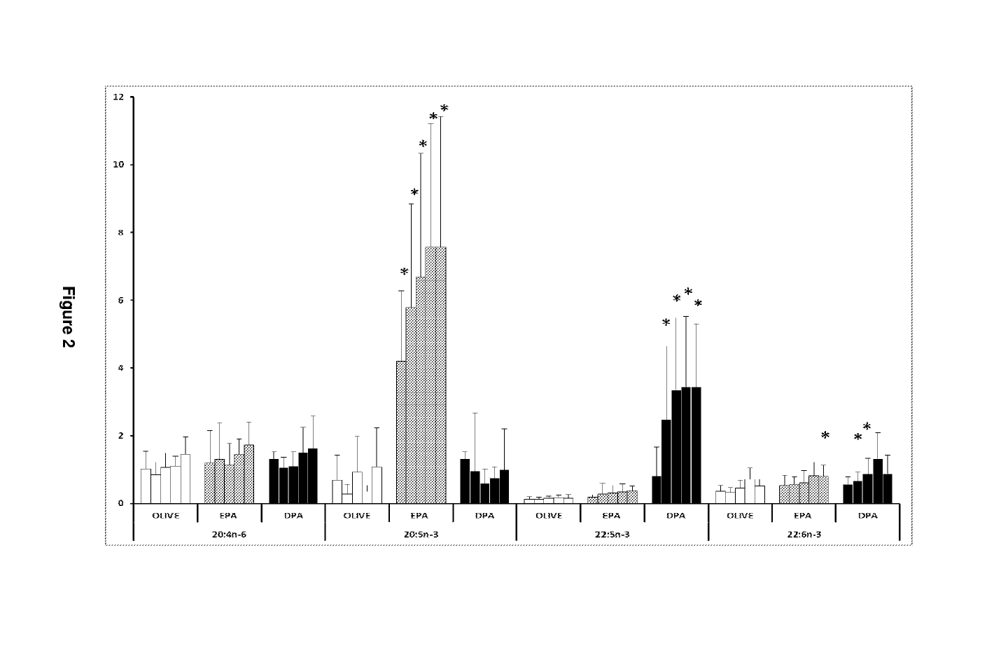

At one to five hours postprandial EPA was significantly higher in the chylomicron TAGs after the breakfast containing EPA than after the breakfasts containing olive oil only or DPA (FIG. 2). Correspondingly the DPA content was significantly higher after the DPA breakfast than after the olive oil meal (2-5 h, p value for the 3 hour difference being 0.06) or after the EPA meal (3-5 h). DPA did not raise the proportion of EPA in chylomicron TAGs. DHA was significantly increased after the DPA breakfast compared to the olive oil breakfast at 2 h and 3 h, and significantly increased after the EPA breakfast compared to the olive oil breakfast at 5 h (FIG. 2).

### Example 3

### Chylomicron Phospholipids

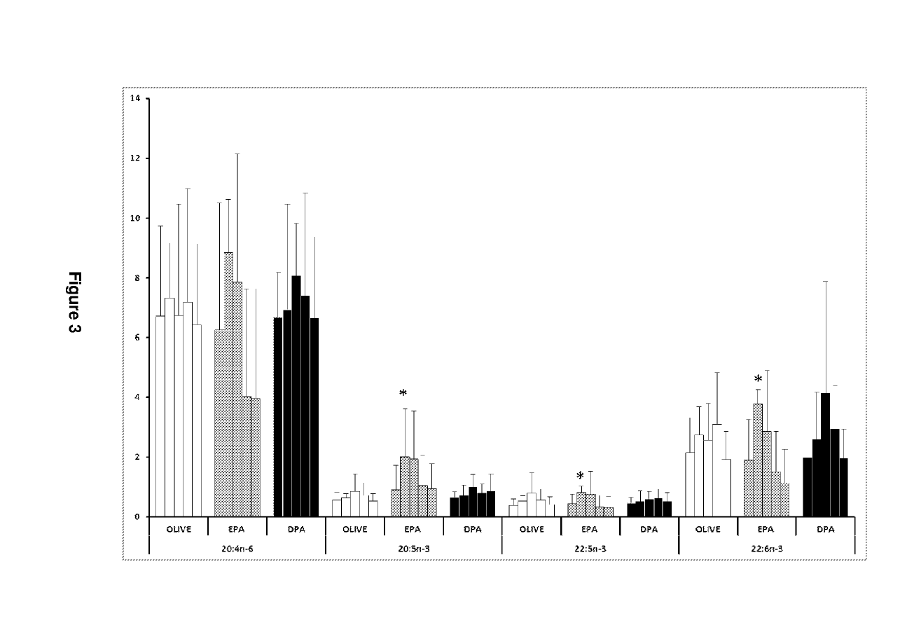

The fatty acid composition of chylomicron phospholipids was less affected by the meal than that of chylomicron TAGs. At 2 h the proportion of EPA was increased after the EPA breakfast compared to the two other breakfasts and at 2 h, the EPA breakfast also increased the amount DPA and DHA compared to the olive oil breakfast (Table 1 and FIG. 3). There were no differences in the prevalences of the polyunsaturated fatty acids at other time points.

TABLE 1
Long chain polyunsaturated fatty acids in the chylomicron
phospholipids at two hours after the meals containing
olive oil, EPA and DPA.

| | FA (mmol/L) | Olive oil | EPA | DPA |
|---|---|---|---|---|
| AA | 20:4n-6 | 7.3 +/− 1.8 | 8.8 +/− 1.8 | 6.9 +/− 3.5 |
| EPA | 20:5n-3 | 0.6 +/− 0.1^{a} | 2.0 +/− 1.6^{b} | 0.7 +/− 0.3^{a} |
| DPA | 22:5n-3 | 0.5 +/− 0.1^{a} | 0.8 +/− 0.2^{b} | 0.5 +/− 0.3^{ab} |
| DHA | 22:6n-3 | 2.7 +/− 0.9^{a} | 3.8 +/− 0.5^{b} | 2.6 +/− 1.6^{ab} |

Values are molar proportions of all chylomicron phospholipid fatty acids +/− standard deviation of 10 subjects. Significant difference p < 0.05 are marked with different letters in each row.

### Example 4

### PUFA Containing Chylomicron TAGs

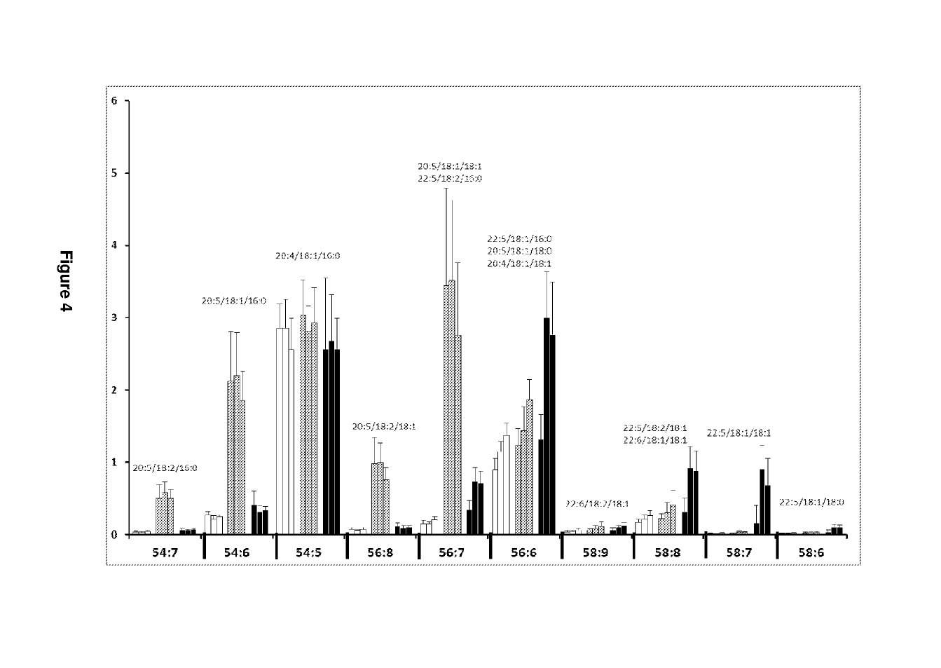

There were significant differences in the concentrations of TAGs containing PUFA between the breakfast groups (FIG. 4). The predominant species contributing to these groups of TAGs were estimated through the use of more extensive multiple-reaction monitoring experiments monitoring the neutral losses of fatty acids. The major species that contained EPA after the EPA breakfast included 20:5/18:1/18:1 and 20:5/18:1/16:0. The overall presence of DPA was lower than that of EPA as seen also from the TAG concentration and fatty acid composition data. The major TAGs containing PUFA after the DPA breakfast were 22:5/18:1/16:0, 22:5/18:2/18:1 and 22:5/18:1/18:1. TAG 54:5, most probably 20:4/18:1/16:0, was detected in equal amounts after all meals.

Although very modest in the overall response, some apparent conversion to DHA was visible in the TAG 58:9 (most probably 22:6/18:2/18:1) as there was significantly more of this TAG after the EPA and DPA breakfasts compared to the olive oil breakfast at the 3 and 5 hour time points. Apart from the PUFA containing TAGs presented in FIG. 4, TAGs 181/18:1/16:0, 18:1/18:1/18:1 and 18:2/18:1/16:0 were abundant TAGs after all meals.

Of the phospholipid species measured, phosphatidyl cholines were the most abundant phospholipid species in chylomicrons followed by inositols, ethanolamines and serines as measured with HPLC-MS/MS. There were no between-breakfast differences in the individual phospholipids or clear increasing or decreasing trends within the measured time points.

No differences were found in chylomicron cholesteryl esters species between breakfasts or between the three measured time points (1, 3, and 5 hr). The most abundant fatty acid in chylomicron cholesteryl esters was 18:2 followed by 16:0, 18:1 and 20:4 in about equal amounts and then by 16:1, 18:3, 20:5 and 22:6.

### Remarks

DPA is an elongated metabolite of EPA and it is one of the intermediate products between EPA and DHA. The present disclosure investigated the postprandial metabolism of pure DPA and EPA in an olive oil containing meal.

The major finding from the study was that just 2 g of n-3 DPA to the 18 g of olive oil almost completely eliminates the incorporation of fatty acids in chylomicrons within five hours. In contrast, this effect was not seen with the addition of EPA.

While not wishing to be bound by theory, the decreased chylomicronemia caused by DPA could be explained if DPA was acting as a pancreatic lipase inhibitor. If DPA did hinder the action of the lipase, the result would be a reduced or slower chylomicronemia and there would be reduced levels of chylomicron TAGs, particularly those with oleic acid (from the 18 g of fed olive oil). Both of these effects were observed in this study. Furthermore, if some of the fat ingested is not thoroughly or efficiently digested by the lipase, some of the fat ingested is not thoroughly or efficiently digested by the lipase, some of the fat would be malabsorbed and lost in the feaces. This hypothesis is supported by the recorded observation that three out of the ten subjects reported diarrea or upset stomach in the three hours following the DPA breakfast.

Another possible explanation relates to the TAG reservoirs that are found to exist in enterocytes (Lambert (2012) Biochim Biophys Acta 1821:721-726).

Other possible mechanisms e.g. ones involving bile salts, absorption into mucosal cells, disruption of TAG synthesis or the packaging of chylomicron, and enhancement of chylomicron clearance are also possible. However, the diarrhea observed by some of the subjects supports effects taking place in the gut rather than in the mucosal cells or blood.

The data presented in this study indicates that the EPA and DPA are metabolised differently postprandial.

1. A pharmaceutical composition comprising n-3 docosapentaenoic acid (DPA) or a derivative thereof in substantially pure form together with a pharmaceutically acceptable carrier or excipient.

2. A composition according to claim 1, wherein the n-3 DPA or derivative thereof comprises at least 10% by weight of the composition.

3. A pharmaceutical composition comprising n-3 docosapentaenoic acid (DPA) or a derivative thereof for use in treating or preventing hypertriglyceridemia or a disorder associated with hypertriglyceridemia in a subject in need thereof.

4. The composition according to claim 3 which treats or prevents post-prandial elevation in blood triglycerides in a subject.

5. A composition according to any one of claims 1 to 4, wherein the n-3 DPA is in free fatty acid form.

6. A composition according to any one of claims 1 to 4, wherein the n-3 DPA is in ethyl ester form.

7. Use of purified n-3 DPA or a derivative thereof, or a pharmaceutical composition according to any one of claims 1 to 6 for treating or preventing hypertriglyceridemia, or a disorder associated with hypertriglyceridemia in a subject in need thereof.

8. Use according to claim 7, wherein the composition treats or prevents post-prandial elevation in blood triglycerides in a subject.

9. The composition according to any one of claims 1 to 6, or a use according to claim 7 or 8, wherein the proportion of EPA in post-prandial triglycerides is not raised.

10. The composition according to any one of claims 1 to 6, or a use according to claim 7 or 8, wherein the composition decreases post prandial chylomicronemia.

11. Use of purified n-3 DPA or a derivative thereof, or a pharmaceutical composition comprising n-3 DPA according to any one of claims 1 to 6 in medicine.

12. Use of purified n-3 DPA or a derivative thereof in the manufacture of a medicament for treating or preventing hypertriglyceridemia or a disorder associated with hypertriglyceridemia in a subject.

13. Use according to claim 12, wherein the medicament treats or prevents post-prandial elevation in blood triglycerides in a subject.

14. A method of reducing fasting triglycerides in a subject in need thereof comprising administering to the subject and effective amount of purified n-3 docosapentaenoic acid (DPA) or derivative thereof, or a pharmaceutical composition according to any one of claims 1 to 6 for a period effective to reduce fasting triglycerides in the subject.

15. A method for treating or preventing hypertriglyceridemia or a disorder associated with hypertriglyceridemia in a subject in need thereof, comprising administering to the subject an effective amount of purified n-3 DPA or derivative thereof, or a pharmaceutical composition according to any one of claims 1 to 6.

16. The method according to claim 15 which treats or prevents post-prandial elevation in blood triglycerides in a subject.

17. Use according to any one of claims 7 to 13, or a method according to any one of claims 14 to 16, wherein the subject has a baseline fasting triglyceride level, from about 400 mg/dl to about 2500 mg/dl.

18. Use according to any one of claims 7 to 13, or a method according to any one of claims claims 14 to 16, wherein the subject being treated has a fasting baseline triglyceride level from about 500 mg/dl to about 2000 mg/dl.

19. The method according to any one of claims 14 to 16, wherein the n-3 DPA is provided in free fatty acid form, in triglyceride form or in ethyl ester form.

20. Use according to any one of claims 7 to 13 or a composition according to any one of claims 1 to 6, or a method according to any one of claims 14 to 16, wherein the n-3 DPA or derivative thereof comprises at least 10% by weight of said n-3 DPA or derivative thereof.

21. Use according to any one of claims 7 to 13 or a composition according to any one of claims 1 to 6, or a method according to any one of claims 14 to 16, wherein the said purified n-3 DPA or composition comprises not more than about 10% eicosapentaenoic acid (EPA) or docosahexaenoic acid (DHA) or a combination of EPA and DHA.

22. Use according to any one of claims 7 to 13 or a composition according to any one of claims 1 to 6, or a method according to any one of claims 14 to 16, wherein the disorder associated with hypertriglyceridemia and/or hypercholesterolemia is selected from (i) a cardiovascular-related disorder, (ii) rheumatoid arthritis, (iii) Raynaud Syndrome, (iv) lupus, (v) menstrual pain (vi) type II diabetes, (vii) obesity, (viii) Crohn's disease, (viv) osteoarthritis, (x) hypothyroidism, (xi) kidney disease and (xii) osteoporosis.

23. Use according to any one of claims 7 to 13 or a composition according to any one of claims 1 to 6, or a method according to any one of claims 14 to 16, wherein the subject is on medication that causes plasma triglycerides to be elevated above normal levels.

24. Use according to any one of claims 7 to 13 or a composition according to any one of claims 1 to 6, or a method according to any one of claims 14 to 16, wherein the subject is taking medication selected from (i) tamoxifen, (ii) steroids, (iii) beta-blockers, (iv) diuretics, (v) estrogen, (vi) oral retinoids and (vii) birth control pills.

25. Use, composition or method according to any preceding claim, wherein the subject is an HIV subject who is on protease inhibitor medication.

26. Use, composition or method according to any preceding claim, wherein the subject is an alcoholic.

27. Use, composition or method according to any preceding claim, wherein the subject has familial lipoprotein lipase deficiency (chylomicronemia syndrome).

28. Use, composition or method according to any preceding claim, wherein the subject has previously been treated with an agent selected from one or more of i) statins, ii) fibrates, iii) nicotinic acid, iv) Lovaza® and v) Vascepa® and has experienced an increase in, or no decrease in triglyceride level, low density lipoprotein cholesterol (LDL-C) level and non-high density lipoprotein cholesterol (HDL-C) level.

29. A method of treating or preventing very high plasma triglyceride levels (e.g. Types IV and V hyperlipidemia) in a subject, comprising administering to the subject an effective amount of purified n-3 DPA or a derivative thereof, or a composition according to any one of claims 1 to 6.

30. Use, composition or method according to any preceding claim, wherein the subject is administered a dose of n-3 DPA or a derivative thereof between 50 mg to about 5000 mg.

31. A weight loss supplement comprising purified n-3 DPA or derivative thereof or a composition comprising n-3 DPA or derivative thereof according to any one of claims 1 to 6.

32. Use of purified n-3 DPA or a derivative thereof, or a composition comprising n-3 DPA or derivative thereof according to any one of claims 1 to 6 in a weight loss supplement for treating or preventing obesity in a subject.

33. A food additive comprising purified n-3 DPA or a pharmaceutical composition according to any one of claims 1 to 6.

34. A food additive according to claim 33 for use in one or more selected from a functional food, nutrient-supplementing food, formula suitable for feeding infants or premature infants, baby foods, foods for expectant or nursing mothers, and geriatric foods.

35. Use of purified n-3 DPA or a derivative thereof as a food additive for supplementing animal feed.

36. An animal feed comprising purified n-3 DPA or a derivative thereof, or a pharmaceutical composition comprising n-3 DPA or a derivative thereof according to any one of claims 1 to 6.

37. A kit comprising purified n-3 DPA or a derivative thereof, or a composition according to any one of claims 1 to 6, packaged together with instructions for use to treat hypertriglyceridemia or a disorder associated with hypertriglyceridemia in a subject.
